(19) United States
(12) Patent Application Publication
(10) Pub. No.: US 2021/0290708 A1
(43) Pub. Date: Sep. 23, 2021

(54) METHODS AND COMPOSITIONS FOR MODULATING MYELOID-DERIVED SUPPRESSOR CELLS

(71) Applicants: NANTCELL, INC., Culver City, CA (US); GLOBEIMMUNE, INC., Louisville, CO (US)

(72) Inventors: Patrick SOON-SHIONG, Culver City, CA (US); Thomas H KING, Denver, CO (US)

(21) Appl. No.: 17/264,624

(22) PCT Filed: Sep. 18, 2019
(86) PCT No.: PCT/US2019/051747

§ 371 Date: Jan. 29, 2021

Related U.S. Application Data
(60) Provisional application No. 62/734,350, filed on Sep. 21, 2018.

(51) Int. Cl. A61K 36/064; A61K 31/716; C12N 1/18; A61P 35/00; A61K 9/00; A61K 39/00

(52) U.S. Cl. CPC A61K 36/064; A61K 31/716; C12N 1/185; A61K 39/0011; A61K 9/0053; A61K 9/0019; A61P 35/00

(57) ABSTRACT

The present invention includes a method and related compositions to modulate myeloid-derived suppressor cell (MDSC) suppressive function, MDSC differentiation, or combinations thereof in a cancer patient by administering a yeast comprising β-glucans to the cancer patient, in which the yeast can be modified to unmask yeast cell wall β-glucans to enhance the effect on MDSCs.

### CROSS-REFERENCE TO RELATED APPLICATIONS

This application claims the benefit of priority under 35 U.S.C. § 119(e) to U.S. Provisional Patent Application No. 62/734,350, filed Sep. 21, 2018. The entire disclosure of U.S. Provisional Patent Application No. 62/734,350 is incorporated herein by reference.

### BACKGROUND OF THE INVENTION

Cancer is a leading cause of death worldwide, and the development of effective therapies for cancer continues to be one of the most active areas of research and clinical development. Although a variety of innovative approaches to treat and prevent cancers have been developed, many cancers continue to have a high rate of mortality and may be difficult to treat or relatively unresponsive to conventional therapies. Emerging immunotherapies including vaccines and/or immune checkpoint inhibitors are welcome additions to the arsenal of anticancer regimens, but some patients either cannot tolerate these treatments at the required doses or experience early disease recurrence because immunosuppressive processes not targeted by the checkpoint inhibitor prevent complete tumor clearance.

Myeloid cells originate from hematopoietic stem cells and mainly exist in bone marrow and lymphatic tissues. Myeloid cells differentiate into macrophages, dendritic cells (DCs), and granulocytes, but these cells do not have a specific hierarchical structure Myeloid cells exist in the tumor microenvironment where they promote tumor angiogenesis, invasion and tumor cell metastasis.

It is well-appreciated that tumors produce a plethora of immune-modulatory factors that constrain antitumor cytotoxic effects mediated by cells of the innate and adaptive immune system. Not only do tumor-derived factors drive angiogenesis for nutrient supply, but they also disrupt the rhythm of differentiation of bone marrow-derived immune cells toward the accumulation and expansion of a heterogeneous population of immature immune-suppressive cells known collectively as myeloid-derived suppressor cells (MDSC). In mice, two main subsets of MDSC have been identified according to their morphology and Gr-I, Ly6C, Ly6G, and CD11b expression: monocytic MDSC (M-MDSC) which resemble monocytes and are Gr-1^{low/int} CD11b+(Ly6c^{high}Ly6G-CD11b+(5) and polymorphonuclear MDSC (PMN-MDSC) which resemble polymorphonuclear granulocytes and are Gr-1^{high}CD1 lb+(Ly6G^{high}Ly6 C^{low}CD11b+). In humans, MDSC lack the Gr-1 homolog and are defined as CD14-HLA-DR-CD11b+CD33+ or CD14+HLA-DR-CD11b+CD33+.

After the identification of MDSCs as key suppressors of effector T cell responses and inducers of T cell tolerance, numerous studies characterized additional roles in cancer including natural killer (NK) cell suppression, regulatory T cell induction, and tumor-associated macrophage formation MDSC-mediated T cell suppression is mainly attributed to the expression of Arginase 1, inducible NO synthase (Inos), reactive oxygen species (ROS), and cystine and cysteine deprivation. A main factor responsible for the accumulation of MDSC in cancer is that MDSC are immature and do not subsequently differentiate to antitumor macrophages and dendritic cells (DC) under the influence of tumor-derived factors. Therefore, the importance of targeting MDSC expansion, suppression, and differentiation in combination with other therapies in cancer well-recognized.

In a preclinical model, chemotherapeutic agents known to directly reduce MDSC include gemcitabine and 5-fluorouracil (5-FU). It was reported that the gemcitabine significantly reduces the number of MDSC in the spleen in tumor-induced mice (Clin Cancer Res 2005; 11: 6713-6721). It is also known that 5-FU significantly reduces MDSC, and it is reported that the degree of reduction by 5-FU is larger than that of the gemcitabine (Int Immunopharmacol. 2017 June:47:173-181.

As another method for suppressing MDSCs, WO 2013/082591 discloses a method of increasing the amount of miR-142 and/or of miR-223 polynucleotide in an MDSC causing its differentiation into a macrophages or DCs.

Yet another method of suppressing MDSCs is disclosed in WO 2011/116299, which includes using bisphosphonate or a chemokine receptor 2 (CCR2) inhibitor as an adjuvant.

To study a natural compound targeting MDSCs, the effect of the immunomodulator particulate β-glucan on MDSC in tumor-bearing animals and non-small cell lung cancer (NSCLC) patients was studied (Albeituni et al. J Immunol 2016; 196:2167-2180). Whole β-glucan particles (WGPs) are microparticles of 1,3-β-glucan extracted from the yeast Saccharomyces cerevisiae, which activate immune cells through the stimulation of the C-type lectin receptor Dectin-1. Previous studies showed that β-glucan treatment activates dendritic cells (DC) and induces therapeutic T cell responses in vivo. WGPs also partially induced the differentiation of M-MDSC to F4/80+CD11e+ cells. The present inventors have identified novel materials and methods involving the use of yeast and related β-glucans to modulate MDSC suppressive function and differentiation.

### SUMMARY OF THE INVENTION

One embodiment of the present invention is a method to modulate MDSC suppressive function, MDSC differentiation, or combinations thereof in a cancer patient. Methods of the invention can have a number of beneficial effects for treating cancer patients, including reducing, limiting or inhibiting MDSC suppression of NK cells MDSC induction of regulatory T cells MDSC maturation to tumor-associated macrophages, and combinations thereof. In other embodiments, the modulation of MDSC suppressive function and differentiation can include an effect selected from inducing polymorphonuclear (PMN) MDSC apoptosis, monocytic (M) MDSC differentiation to antigen presenting cells (APCs), and combinations thereof. In other embodiments, the administration of yeast induces apoptosis of PMN-MDSCs or induces differentiation of M-MDSC to functionally active APCs.

The method includes administering a yeast to a cancer patient, where the yeast comprises β-glucans. The yeast are a rich source of β-glucans that can impair the immunosuppressive function of MDSCs by one or more of the above mechanisms. In some embodiments, the yeast can be modified to unmask yeast cell wall β-glucans, such as by being genetically engineered to reduce the activity of enzymes that elongate and/or branch yeast cell wall mannosyl groups or to reduce the enzymatic activity of a Golgi mannosyltransferase complex. In these embodiments, the yeast can be selected from the group consisting of a Mnn9 deleted strain, a Mnn10 deleted strain and a doubly deleted Mnn9/Mnn10 strain of yeast. In addition, or alternatively, the yeast can be genetically engineered to increase the enzymatic activity of 1,3 β-glucan synthase. A yeast modified to unmask yeast cell wall β-glucans can also be a yeast that has been treated to remove yeast cell wall glycosyl groups, such as a yeast that has been contacted with the enzymes PNGaseF (Peptide-N-Glycosidase F) or Endoglycosidase H (ENDO-H) which remove N-linked oligosaccharides from glycoproteins, or alpha-mannosidase which removes O-linked oligosaccharides from cell wall glycoproteins. Further, such a yeast can be one that has been cultured under neutral pH conditions, such as in medium where the media was maintained at a pH level of between 5.5 and 8 for at least 50% of time that the yeast were being cultured.

In embodiments, the yeast can be selected from Saccharomyces, Candida, Cryptococcus, Hansenula, Kluyveromyces, Pichia, Rhodotorula, Schizosaccharomyces and Yarrowia, and in some embodiments, the yeast is Saccharomyces. In other embodiments the yeast can be selected from Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica, and in some embodiments is Saccharomyces cerevisiae.

In various embodiments, the yeast can be administered to the cancer patient by routes of administration including intravenous, intraperitoneal, subcutaneous, intradermal, intranodal, intramuscular, transdermal, inhaled, intranasal, oral, intratumoral, intraocular, intraarticular, intracranial, and intraspinal. In other embodiments, administration is by oral administration or by intratumoral administration.

Patients suitable for being treated by methods and compositions of the invention include cancer patients having a cancer selected from lung cancer, breast cancer, triple negative breast cancer (TNBC), colorectal cancer, liver cancer, stomach cancer, colon cancer, non-small cell lung cancer (NSCLC), bone cancer, malignant chordoma, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladder cancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary central nervous system (CNS) tumor, spinal cord tumor, brainstem glioma, and pituitary adenoma. In other embodiments, the patients have a cancer selected from ovarian, colorectal, pancreatic, urothelial, merkel, melanoma, NSCLC, head and neck squamous cell carcinoma, and triple negative breast cancer.

In other embodiments of the invention, methods of the present invention include administration of a yeast where the yeast is a yeast vehicle selected from a whole yeast, a yeast spheroplast, a yeast cytoplast, a yeast ghost, and a subcellular yeast particle, and the yeast is in an immunogenic composition further including at least one antigen and/or neoantigen that is heterologous to the yeast. In such embodiments, the antigen can be from a tumor or cancer selected from melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, leukemias, lymphomas, primary hepatic cancers, lung cancers, pancreatic cancers, gastrointestinal cancers, renal cell carcinomas, hematopoietic neoplasias and metastatic cancers thereof. In more particular embodiments, the antigen can be selected from carcinoembryonic antigen (CEA), carcinoembryonic antigen peptide 1 (CAP-1), carcinoembryonic antigen peptide 1-6D (CAP-1-6D), melanoma antigen recognized by T cells 1 (MART-1), melanoma-associated antigen 1 (MAGE-1), melanoma-associated antigen 3 (MAGE-3), GAGE, glycoportien 100 (GP-100), mucin 1 (MUC-1), mucin 2 (MUC-2), point mutated Ras oncoprotein, tumor protein p53 (p53), point mutated p53, prostate-specific membrane antigen (PSMA), tyrosinase, tyrosinase related protein 1 (TRP-1; also referred to as gp75), New York esophageal squamous cells carcinoma 1 (NY-ESO-1), tyrosinase related protein 2 (TRP-2), tumor associated glycoprotein 72 (TAG72), KSA, cancer antigen 125 (CA-125), prostate-specific antigen (PSA), human epidermal growth factor receptor 2 c-erb-B2 (HER-2/neu/c-erb/B2), epidermal growth factor receptor (EGFR), human telomerase reverse transcriptase (hTERT), tumor protein 73 (p′73), serine/threonine-protein kinase B-Raf (B-RAF), adenomatous polyposis coli (APC), Myc, von Hippel-Lindau protein (VHL), retinoblastoma protein 1 (Rb-1), retinoblastoma protein 2 (Rb-2), androgen receptor (AR), mothers against decapentaplegic homolog 4 (Smad4), multi-drug resistance 1 (MDR1), FMS-like tyrosine kinase 3 (Flt-3), breast cancer gene 1 (BRCA-1), breast cancer gene 2 (BRCA-2), Bcr-Abl, pax3-fkhr, ews-fli-1, Brachyury, human endogenous retrovirus subfamily H (HERV-H), human endogenous retrovirus subfamily K (HERV-K), TWIST, Mesothelin, new gene expressed in prostate (NGEP), and modifications and epitopes of such antigens.

Other embodiments of the invention include a pharmaceutical composition, that includes a yeast comprising β-glucans and a pharmaceutically acceptable excipient. In some embodiments, the yeast is modified to unmask yeast cell wall β-glucans.

The yeast in the pharmaceutical composition can be genetically engineered to reduce the enzymatic activity of enzymes that elongate and/or branch yeast cell wall mannosyl groups; genetically engineered to reduce the enzymatic activity of a Golgi mannosyltransferase complex; and/or genetically engineered to increase the enzymatic activity of 1,3 β-glucan synthase. Such yeast can be selected from a Mnn9 deleted strain, a Mnn10 deleted strain and a Mnn9/Mnn10 doubly deleted strain of yeast. In addition, the yeast can have been treated to remove yeast cell wall glycosyl groups, such as by contacting the yeast with PNGaseF or ENDO-h to remove N-linked mannosyl groups from cell wall glycoproteins or alpha-mannosidase which removes O-linked oligosaccharides from cell wall glycoproteins.

Suitable pharmaceutically acceptable excipients can include an isotonic buffer that is tolerated by a host or host cell. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, glycerol or dextran.

### BRIEF DESCRIPTION OF THE DRAWINGS

FIG. 1 shows the various layers of the fungal cell wall.

### DETAILED DESCRIPTION OF THE INVENTION

This invention generally relates to a method to modulate myeloid-derived suppressor cells (MDSCs) suppressive function and differentiation in a cancer patient by administering a yeast to a cancer patient, wherein the yeast comprises β-glucans. This method takes advantage of β-glucans in the yeast cell wall to offset the effects of MDSCs and to support the patient's ability to mount an improved immune response against a cancer. Another embodiment of the invention relates to a pharmaceutical composition, comprising a yeast modified to unmask yeast cell wall β-glucans and a pharmaceutically acceptable excipient.

MDSCs function to suppress immunity by inhibiting various T cell functions including the antitumor activity of cytotoxic T lymphocytes. MDSCs can have a positive function of suppressing excessive immune responses such as in autoimmune conditions, but they can also have a negative function of generating or deteriorating diseases or interrupting proper treatment by suppressing immunity in a situation where immune response is beneficial. For example, MDSCs are largely increased in tumor or cancer patients, and this significantly reduces the beneficial effects of administering cancer immunotherapy, such as cancer vaccines. In such a situation, when the number of MDSCs is effectively reduced or the immunosuppressive effects of MDSCs are offset by activation of the immune system, cancer treatment can be more effective.

Modulation of MDSCs' suppressive function and differentiation refers to a beneficial effect of yeast comprising β-glucans on the function or status of MDSCs in the management of disease in a cancer patient. For example, administration of yeast can have an effect selected from reducing, limiting or inhibiting MDSC suppression of NK cells, MDSC induction of regulatory T cells, MDSC maturation to tumor-associated macrophages, and combinations thereof. The suppression of MDSCs can comprise an effect selected from inducing polymorphonuclear (PMN) MDSC apoptosis, monocytic (M) MDSC differentiation to antigen presenting cells (APCs), and combinations thereof.

Methods of the present invention to modulate MDSCs' suppressive function and differentiation can be used in the treatment of cancers, including by preventing emergence of cancers, arresting progression of cancers or eliminating cancers. More particularly, such methods can be used to inhibit or delay the development of cancer, and/or to prevent, inhibit or delay tumor migration and/or tumor invasion of other tissues (metastases) and/or to generally prevent or inhibit progression of cancer in an individual. Such methods can also be used to ameliorate at least one symptom of the cancer, such as by reducing tumor burden in the individual; inhibiting tumor growth in the individual; increasing survival of the individual; and/or preventing, inhibiting, reversing or delaying progression of the cancer in the individual.

### Yeast in General

These methods and compositions herein are broadly applicable to all yeast. Yeast are unicellular microorganisms that belong to one of three classes: Ascomycetes, Basidiomycetes and Fungi Imperfecti. While pathogenic yeast strains, or nonpathogenic mutants thereof can be used in accordance with the present invention, in one aspect, nonpathogenic yeast strains are used. Examples of nonpathogenic yeast strains include Saccharomyces, Candida, Cryptococcus, Hansenula, Kluyveromyces, Pichia, Rhodotorula, Schizosaccharomyces and Yarrowia. In one aspect, Saccharomyces, Candida, Hansenula, Pichia and Schizosaccharomyces are used. In yet other aspects, Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica are used. It is understood the invention is not limited to the species listed above and that one of skill in the art can apply the teachings herein any type of yeast. In another aspect, Saccharomyces cerevisiae (S. cerevisiae) is used to practice the methods of the invention. S. cerevisiae is preferred due to it ease for molecular manipulation and being “Generally Recognized As Safe” or “GRAS” for use as food additives (GRAS, FDA proposed Rule 62FR18938, Apr. 17, 1997).

According to the present invention, the yeast (also referred to as a “yeast vehicle”) is selected from the group consisting of a whole yeast, a yeast spheroplast, a yeast cytoplast, a yeast ghost, and a subcellular yeast particle.

The major components of yeast cell walls are polysaccharides and glycoproteins. In Saccharomyces cerevisiae, for example, the cell wall contains β(1→3)-D-glucan, β(1→6)-D-glucan, chitin, and mannoprotein(s). The polysaccharides appear to have a structural function, whereas the mannoprotein(s) may act as “filler” and are important for the permeability of the cell wall. Thus, yeast useful in the present invention can have naturally occurring β-glucans in their cell wall that function to modulate MDSC suppressive function and differentiation. In addition, the yeast can be manipulated to increase the amount of or availability of β-glucans to provide improved modulation of MDSC suppressive function and differentiation.

### Yeast Modified to Unmask Beta-Glucans

The yeast that is administered in the invention can be modified to unmask or expose the yeast cell wall β-glucans thereby making the β-glucans more available to modulate MDSC suppressive function and differentiation. Such modifications can include yeast that are genetically engineered to reduce the enzymatic activity of enzymes that elongate and/or branch yeast cell wall mannosyl groups, as well as yeast that are genetically engineered to reduce the enzymatic activity of a Golgi mannosyltransferase complex. Such modifications can also include yeast that are genetically engineered to increase the activity of enzymes that produce β-glucans in yeast. For example, in one embodiment, the yeast can be a deletion mutant of mannan polymerase complex subunit MNN9 (Mnn9 del strain or Mnn9 deletion strain), a deletion mutant of mannan polymerase complex subunit MNN10 (Mnn10 del strain or Mnn10 deletion strain), or a deletion mutant of both mannan polymerase complex subunit MNN9 and mannan polymerase complex subunit MNN10 (Mnn9/Mnn10 del strain or Mnn9/Mnn10 deletion strain). In another embodiment, the yeast can be genetically modified to increase the enzymatic activity of 1,3 β-glucan synthase. Another embodiment of a modified yeast is one in which yeast have been exposed to peptide-N-glycosidase F (PNGaseF), a amidase, which cleaves between the innermost GlcNAc and asparagine residues of high mannose, hybrid, and complex oligosaccharides from N-linked glycoproteins. Application of PNGaseF is the most effective enzymatic method for removing almost all N-linked oligosaccharides from glycoproteins.

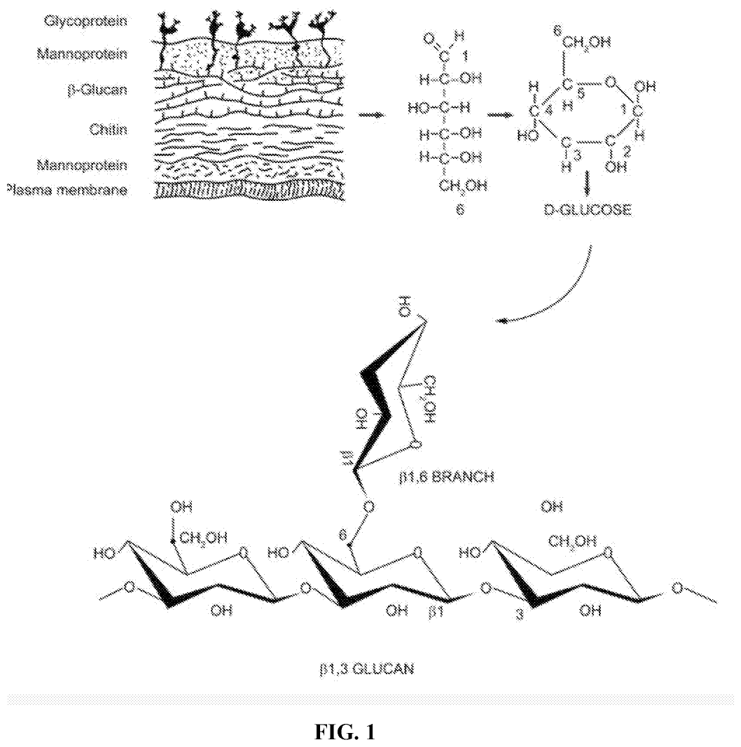

The effect of unmasking yeast cell wall β-glucans is illustrated by reference to FIG. 1 which shows the various layers of the yeast cell wall. β-glucan is one of the key components of the fungal cell wall. The basic subunit of the fungal β-glucan is β-D-glucose linked to one another by 1→3 glycosidic chain with 1→6 glycosidic branches. The length and branches of the β-glucan from various fungi are widely different.

In a further embodiment, the yeast modified to unmask or expose yeast cell wall β-glucans can have been cultured under conditions of neutral pH. This process results in a yeast having a more porous cell wall in which certain cell wall proteins are much more readily exposed and available. Such yeast are believed to be more effective than other yeast at modulating MDSC suppressive function and differentiation. The details of this process are described in U.S. Pat. No. 9,549,970. Briefly, yeast is cultured such that the pH level of the medium does not drop below pH 5.5. In some cases, the drop below pH 5.5 is not more than 5 minutes. In other cases, the drop below pH 5.5 is not more than 10, 20, 30, 40, 50 or 60 minutes. In other cases, the drop below pH 5.5 is not more than 1 hour. In another aspect, yeast is cultured such that the pH level of the medium does not drop below 5.0. In some cases, the drop below pH 5.0 is not more than 5 minutes. In other cases, the drop below pH 5.0 is not more than 10, 20, 30, 40, 50 or 60 minutes. In other cases, the drop below pH 5.0 is not more than 1 hour. As such, the longer time the yeast are grown in a medium that is at least pH 5.5 or above, the better the results will be in terms of obtaining yeast with desirable characteristics described above.

In one aspect, the use of neutral pH methods to grow yeast cells means that the yeast cells are grown in neutral pH for at least 50% of the time that the yeast are in culture. The yeast can be grown at neutral pH for at least 60% of the time they are in culture, at least 70% of the time they are in culture, at least 80% of the time they are in culture, and at least 90% of the time they are in culture. In another aspect, growing yeast at neutral pH includes culturing yeast cells for at least five minutes at neutral pH, at least 15 minutes at neutral pH, at least one hour at neutral pH, at least two hours, or at least three hours or longer.

### Cancer Patients

Methods of the present invention include administration of a yeast to a cancer patient, which can include any animal, including any vertebrate, and particularly to any member of the Vertebrate class, Mammalia, including, without limitation, primates, rodents, livestock and domestic pets. Livestock include mammals to be consumed or that produce useful products (e.g., sheep for wool production). Mammals to treat or protect utilizing the invention include humans, non-human primates, dogs, cats, mice, rats, goats, sheep, cattle, horses and pigs. The term “patient” can be used interchangeably with the term “animal”, “subject” or “individual”.

Such cancer patients can have a cancer and/or a tumor selected from the group consisting of lung cancer, breast cancer, triple negative breast cancer (TNBC), colorectal cancer, liver cancer, stomach cancer, colon cancer, non-small cell lung cancer (NSCLC), bone cancer, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladder cancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary CNS tumor, spinal cord tumor, brainstem glioma, and pituitary adenoma, but is not limited thereto.

Some cancers are strongly influenced by MDSCs and thus, patients affected by these cancers will experience a greater benefit due to the modulation of MDSC suppressive function and differentiation according to the invention. Such a benefit will synergize well with the use of yeast vaccines and viral vector vaccines which have the characteristic of inducing a T-cell response, as antigen presentation is improved and other immunosuppressive effects of MDSCs including Treg recruitment is alleviated. These cancers include but are not limited to ovarian, colorectal, pancreatic, urothelial, merkel, melanoma, NSCLC, head and neck squamous cell carcinoma, and triple negative breast cancer (TNBC). TNBC is defined by the absence of estrogen and progesterone receptor expression and the lack of human epidermal growth factor receptor-2 (HER2) overexpression and/or amplification. Because of the absence of HER-2 receptor expression and hormone receptors, TNBC remains a disease refractory to advancements in targeted therapies and is an especially high unmet need indication. Thus, in a further embodiment of the invention, the cancer patient has a cancer selected from the group consisting of ovarian, colorectal, pancreatic, urothelial, merkel, melanoma, NSCLC, head and neck squamous cell carcinoma, and triple negative breast cancer (TNBC).

In some aspects of the invention, the cancer patient may have stage I, stage II, stage III, or stage IV cancer. In other aspects, use of the yeast composition reduces, eliminates or slows or arrests the growth of tumors, which can result in reduction in tumor burden in the individual, inhibition of tumor growth, and/or increased survival of the individual.

### Steps of Administering

The present invention includes the delivery (administration) of a yeast to a subject or individual in a therapeutic composition of the invention. The administration process can be performed ex vivo or in vivo but is typically performed in vivo. Ex vivo administration refers to performing part of the regulatory step outside of the patient, such as administering a composition of the present invention to a population of cells removed from a patient and returning the cells to the patient. The composition of the present invention can then be returned to a patient, or administered to a patient, by any suitable mode of administration.

Administration of the yeast and/or composition of the invention can be systemic, mucosal and/or proximal to the location of the target site (e.g., near a site of a tumor). Suitable routes of administration will be apparent to those of skill in the art, depending on the type of cancer to be prevented or treated and/or the target cell population or tissue. Various acceptable methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, aural, intranasal, oral, intratumoral, pulmonary administration, impregnation of a catheter, and direct injection into a tissue. In one aspect, routes of administration include: intravenous, intraperitoneal, subcutaneous, intradermal, intranodal, intramuscular, transdermal, inhaled, intranasal, oral, intratumoral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992). In one aspect, a yeast and/or composition of the invention is administered subcutaneously. In one aspect, the yeast and/or composition of the invention is administered directly into a tumor milieu. In one aspect, the yeast and/or composition is administered to the cancer patient by oral administration. In one aspect, the yeast and/or composition of the invention is administered to the cancer patient by intratumoral administration. MDSCs are located in tumors and thus, it is believed that local tumor targeted administration of the yeast will have a more focused effect.

In general, a suitable single dose of a yeast and/or composition of the invention is a dose that is capable of effectively modulating MDSCs suppressive function and differentiation in a cancer patient, when administered one or more times over a suitable time period. For example, in one embodiment, a single dose of a yeast of the present invention is from about 1×10⁵ to about 5×10′ yeast cell equivalents per kilogram body weight of the organism being administered the composition. One Yeast Unit (Y.U.) is 1×10′ yeast cells or yeast cell equivalents. In one aspect, a single dose of a yeast of the present invention is from about 0.1 Y.U. (1×10⁶ yeast cells or yeast cell equivalents) to about 100 Y.U. (1×10⁹ cells) per dose (i.e., per organism), including any interim dose, in increments of 0.1×10⁶ cells (i.e., 1.1×10⁶, 1.2×10⁶, 1.3×10⁶ . . . ). In one embodiment, a suitable dose includes doses between 1 Y.U. and 40 Y.U. and in one aspect, between 10 Y.U. and 40 Y.U. or between 10 Y.U. and 80 Y.U. In one embodiment, the doses are administered at different sites on the individual but during the same dosing period. For example, a 40 Y.U. dose may be administered by injecting 10 Y.U. doses to four different sites on the individual during one dosing period. The invention includes administration of an amount of the yeast immunotherapy composition (e.g., 1, 2, 3, 4, 5, 6, 7, 8, 9 10, 11, 12, 13, 14,15, 16, 17, 18, 19, 20 Y.U. or more) at 1, 2, 3, 4, 5, 6, 7, 8, 9, 10, or more different sites on an individual to form a single dose.

“Boosters” or “boosts” of a therapeutic composition are administered, for example, when the effect on MDSCs has waned. Boosters can be administered about 1, 2, 3, 4, 5, 6, 7, or 8 weeks apart, or monthly, bimonthly, quarterly, annually, and/or in a few or several year increments after the original administration, depending on the status of the individual being treated and the goal of the therapy at the time of administration (e.g., prophylactic, active treatment, maintenance). In one embodiment, the doses are administered weekly or biweekly for 1, 2, 3, 4, 5, 6, 7, 8, 9, 10 or more doses, followed by biweekly or monthly doses as needed to achieve the desired preventative or therapeutic treatment for cancer. Additional boosters can then be given at similar or longer intervals (months or years) as a maintenance or remission therapy, if desired.

Yeast with Cancer Antigens

In one embodiment of the invention, the method of the invention also includes administering to a cancer patient a yeast in a composition that includes an antigen heterologous to the yeast and useful for cancer immunotherapy. The yeast in the yeast and antigen composition can be (i) a yeast that is modified to unmask yeast cell wall β-glucans or (ii) a yeast that has not been so modified but is combined with a yeast that is modified to unmask yeast cell wall β-glucans. In both cases, the method has the dual effect of modulating MDSC suppressive function and differentiation (due to the unmasked yeast cell wall β-glucans) and of eliciting an immune response against the antigen of interest.

According to the present invention, the general use herein of the term “antigen” refers: to any portion of a protein (peptide, partial protein, full-length protein), wherein the protein is naturally occurring or synthetically derived, to a cellular composition (whole cell, cell lysate or disrupted cells), to a microorganism or cells (whole microorganism, lysate or disrupted cells) or to a carbohydrate, or other molecule, or a portion thereof. An antigen may, in some embodiments, elicit an antigen-specific immune response (e.g., a humoral and/or a cell-mediated immune response) against the same or similar antigens that are encountered by an element of the immune system (e.g., T cells, antibodies). The term “cancer antigen” can be used interchangeably herein with the terms “tumor-specific antigen”, “tumor-associated antigen”, “cancer-associated target” or “tumor-associated target” or “neoantigen”.

An antigen can be as small as a single epitope, or larger, and can include multiple epitopes. As such, the size of an antigen can be as small as about 5-16 amino acids (e.g., a small peptide) and as large as: a domain of a protein, a partial protein (peptide or polypeptide), a full-length protein, including a multimer and fusion protein, chimeric protein, or agonist protein or peptide. In addition, antigens can include carbohydrates.

When referring to stimulation of an immune response, the term “immunogen” is a subset of the term “antigen”, and therefore, in some instances, can be used interchangeably with the term “antigen”. An immunogen, as used herein, describes an antigen which elicits a humoral and/or cell-mediated immune response (i.e., is immunogenic), such that administration of the immunogen to an individual in the appropriate context (e.g., as part of a yeast-based immunotherapy composition) elicits or induces an antigen-specific immune response against the same or similar antigens that are encountered by the immune system of the individual.

An “immunogenic domain” of a given antigen can be any portion, fragment or epitope of an antigen (e.g., a peptide fragment or subunit or an antibody epitope or other conformational epitope) that contains at least one epitope that acts as an immunogen when administered to an animal. For example, a single protein can contain multiple different immunogenic domains. Immunogenic domains need not be linear sequences within a protein, such as in the case of a humoral immune response.

An epitope is defined herein as a single immunogenic site within a given antigen that is sufficient to elicit an immune response. Those of skill in the art will recognize that T cell epitopes are different in size and composition from B cell epitopes, and that epitopes presented through the Class I WIC pathway differ from epitopes presented through the Class II WIC pathway. Epitopes can be linear sequence or conformational epitopes (conserved binding regions).

The antigens contemplated for use in this invention include any cancer antigen against which it is desired to elicit an immune response, and in particular, include any cancer antigen for which a therapeutic immune response against such antigen would be beneficial to an individual. These antigens can be native antigens or genetically engineered antigens which have been modified in some manner (e.g., sequence change or generation of a fusion protein). It will be appreciated that in some embodiments (i.e., when the antigen is expressed by the yeast from a recombinant nucleic acid molecule), the antigen can be a protein, any epitope or any neoepitope or immunogenic domain thereof, a fusion protein, or a chimeric protein, rather than an entire cell or microorganism.

Antigens useful in one or more immunotherapy compositions of the invention include any cancer or tumor-associated antigen. In one aspect, the antigen includes an antigen associated with a preneoplastic or hyperplastic state. The antigen may also be associated with, or causative of cancer. Such an antigen may be tumor-specific antigen, tumor-associated antigen (TAA) or tissue-specific antigen, epitope thereof, and epitope agonist thereof. Cancer antigens include, but are not limited to, antigens from any tumor or cancer, including, but not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, leukemias, lymphomas, primary hepatic cancers, lung cancers, pancreatic cancers, gastrointestinal cancers (including colorectal cancers), renal cell carcinomas, hematopoietic neoplasias and metastatic cancers thereof.

Suitable cancer antigens include but are not limited to carcinoembryonic antigen (CEA), MUC-1, MUC-2, point mutated Ras oncoprotein, normal and point mutated p53 oncoproteins (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987), TRP-1 (gp75) (Cohen et al Nucleic Acid Res. 18:2807-2808, 1990; U.S. Pat. No. 5,840,839), NY-ESO-1 (Chen et al PAS 94: 1914-1918, 1997), TRP-2 (Jackson et al EMBOJ, 11:527-535, 1992), TAG72, KSA, CA-125, PSA, HER-2/neu/c-erb/B2, (U.S. Pat. No. 5,550,214), EGFR, hTERT, p′73, B-RAF, adenomatous polyposis coli (APC), Myc, von Hippel-Lindau protein (VHL), Rb-1, Rb-2, androgen receptor (AR), Smad4, MDR1, Flt-3, BRCA-1, BRCA-2, Bcr-Abl, pax3-fkhr, ews-fli-1, Brachyury, HERV-H, HERV-K, TWIST, Mesothelin, NGEP, modifications of such antigens and tissue specific antigens, splice variants of such antigens, and/or epitope agonists of such antigens. Other cancer antigens are known in the art. Other cancer antigens may also be identified, isolated and cloned by methods known in the art such as those disclosed in U.S. Pat. No. 4,514,506. Cancer antigens may also include one or more growth factors and splice variants of each.

Cancer antigens can also be non-self-neoantigens created by genetic mutations in normal cellular proteins within tumors. These new mutations can create novel T cell neoepitopes and as such represent a vast array of potential new targets for yeast-based immunotherapy.

Methods of producing yeast and expressing, combining and/or associating yeast with antigens and/or other proteins and/or agents of interest to produce yeast-based and particularly, yeast-based immunotherapy compositions are contemplated by the invention.

According to the present invention, the term “yeast-antigen complex” is used generically to describe any association of a yeast with an antigen and can be used interchangeably with “yeast-based immunotherapy composition” when such composition is used to elicit an immune response as described above. Such association includes expression of the antigen by the yeast (a recombinant yeast), introduction of an antigen into a yeast, physical attachment of the antigen to the yeast, and mixing of the yeast and antigen together, such as in a buffer or other solution or formulation. These types of complexes are described in detail below.

In one embodiment, a yeast cell used to prepare the yeast is transfected with a heterologous nucleic acid molecule encoding a protein (e.g., the antigen) such that the protein is expressed by the yeast cell. Such a yeast is also referred to herein as a recombinant yeast. The yeast cell can then be formulated with a pharmaceutically acceptable excipient and administered directly to a patient, stored for later administration, or loaded into a dendritic cell as an intact cell. The yeast cell can also be killed, or it can be derivatized such as by formation of yeast spheroplasts, cytoplasts, ghosts, or subcellular particles, any of which may be followed by storing, administering, or loading of the derivative into the dendritic cell. Yeast spheroplasts can also be directly transfected with a recombinant nucleic acid molecule (e.g., the spheroplast is produced from a whole yeast, and then transfected) in order to produce a recombinant spheroplast that expresses the antigen. Yeast cells or yeast spheroplasts that recombinantly express the antigen(s) may be used to produce a yeast comprising a yeast cytoplast, a yeast ghost, or a yeast membrane particle or yeast cell wall particle, or fraction thereof.

In general, the yeast and antigen(s) and/or other agents can be associated by any technique described herein. In one aspect, the yeast was loaded intracellularly with the antigen(s) and/or agent(s). In another aspect, the antigen(s) and/or agent(s) was covalently or non-covalently attached to the yeast. In one aspect, antigen(s) and/or agents (including immuno-modulating antibodies) are expressed on the yeast cell wall covalently by fusing them in frame to outer cell wall proteins. In yet another aspect, the yeast and the antigen(s) and/or agent(s) were associated by mixing. In another aspect, and in one embodiment, the antigen(s) and/or agent(s) are expressed recombinantly by the yeast or by the yeast cell or yeast spheroplast from which the yeast was derived.

A number of antigens and/or other proteins to be produced by a yeast of the present invention is any number of antigens and/or other proteins that can be reasonably produced by a yeast. The number of antigens (including polyepitopes, having multiple neoantigens and/or neoepitopes) can range from at least one to about 50, from about 2 to about 40, from about 2 to about 30, from about 2 to about 20, or from about 2 to about 10.

Expression of an antigen or other protein in a yeast of the present invention is accomplished using techniques known to those skilled in the art. Briefly, a nucleic acid molecule encoding at least one desired antigen or other protein is inserted into an expression vector in such a manner that the nucleic acid molecule is operatively linked to a transcription control sequence in order to be capable of effecting either constitutive or regulated expression of the nucleic acid molecule when transformed into a host yeast cell. Nucleic acid molecules encoding one or more antigens and/or other proteins can be on one or more expression vectors operatively linked to one or more expression control sequences. Particularly important expression control sequences are those which control transcription initiation, such as promoter and upstream activation sequences. Any suitable yeast promoter can be used in the present invention and a variety of such promoters are known to those skilled in the art. Promoters for expression in Saccharomyces cerevisiae include, but are not limited to, promoters of genes encoding the following yeast proteins: alcohol dehydrogenase I (ADH1) or II (ADH2), CUP1, phosphoglycerate kinase (PGK), triose phosphate isomerase (TPI), translational elongation factor EF-1 alpha (TEF2), glyceraldehyde-3-phosphate dehydrogenase (GAPDH; also referred to as TDH3, for triose phosphate dehydrogenase), galactokinase (GAL1), galactose-1-phosphate uridyl-transferase (GAL7), UDP-glucose-4 epimerase (GAL10), cytochrome cl (CYC1), Sec? protein (SECT) and acid phosphatase (PH05), including hybrid promoters such as ADH2/GAPDH and CYC1/GAL10 promoters, and including the ADH2/GAPDH promoter, which is induced when glucose concentrations in the cell are low (e.g., about 0.1 to about 0.2 percent), as well as the CUP1 promoter and the TEF2 promoter. Likewise, a number of upstream activation sequences (UASs), also referred to as enhancers, are known. Upstream activation sequences for expression in Saccharomyces cerevisiae include, but are not limited to, the UASs of genes encoding the following proteins: PCK1, TPI, TDH3, CYC1, ADH1, ADH2, SUC2, GAL1, GAL7 and GAL10, as well as other UASs activated by the GAL4 gene product, with the ADH2 UAS being used in one aspect. Since the ADH2 UAS is activated by the ADR1 gene product, it may be preferable to overexpress the ADR1 gene when a heterologous gene is operatively linked to the ADH2 UAS. Transcription termination sequences for expression in Saccharomyces cerevisiae include the termination sequences of the α-factor, GAPDH, and CYC1 genes.

Transcription control sequences to express genes in methyltrophic yeast include the transcription control regions of the genes encoding alcohol oxidase and formate dehydrogenase.

Transfection of a nucleic acid molecule into a yeast cell according to the present invention can be accomplished by any method by which a nucleic acid molecule can be introduced into the cell and includes, but is not limited to, lithium acetate/polyethylene glycol-mediated transformation, diffusion, active transport, bath sonication, electroporation, microinjection, lipofection, adsorption, and protoplast fusion. Transfected nucleic acid molecules can be integrated into a yeast chromosome or maintained on extrachromosomal vectors using techniques known to those skilled in the art. Examples of yeast carrying such nucleic acid molecules are disclosed in detail herein. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using techniques known to those skilled in the art to produce cytoplast, ghost or subcellular yeast membrane extract or fractions thereof containing desired antigens or other proteins.

Effective conditions for the production of recombinant yeast and expression of the antigen and/or other protein by the yeast include an effective medium in which a yeast strain can be cultured. An effective medium is typically an aqueous medium comprising assimilable carbohydrate, nitrogen and phosphate sources, as well as appropriate salts, minerals, metals and other nutrients, such as vitamins and growth factors. The medium may comprise complex nutrients or may be a defined minimal medium. Yeast strains of the present invention can be cultured in a variety of containers, including, but not limited to, bioreactors, Erlenmeyer flasks, test tubes, microtiter dishes, and Petri plates. Culturing is carried out at a temperature, pH and oxygen content appropriate for the yeast strain. Such culturing conditions are well within the expertise of one of ordinary skill in the art (see, for example, Guthrie et al. (eds.), 1991, Methods in Enzymology, vol. 194, Academic Press, San Diego). For example, under one protocol, liquid cultures containing a suitable medium can be inoculated using cultures obtained from starter plates and/or starter cultures of Yeast-MUC1 immunotherapy compositions and are grown for approximately 20 h at 30° C., with agitation at 250 rpm. Primary cultures can then be expanded into larger cultures as desired. Protein expression from vectors with which the yeast were transformed (e.g., MUC1 expression) may be constitutive if the promoter utilized is a constitutive promoter, or may be induced by addition of the appropriate induction conditions for the promoter if the promoter utilized is an inducible promoter (e.g., copper sulfate in the case of the CUP1 promoter). In the case of an inducible promoter, induction of protein expression may be initiated after the culture has grown to a suitable cell density, which may be at about 0.2 Y.U./ml or higher densities.

One non-limiting example of a medium suitable for the culture of a yeast immunotherapy composition of the invention is U2 medium. U2 medium comprises the following components: 15 g/L of glucose, 6.7 g/L of Yeast nitrogen base containing ammonium sulfate, and 0.04 mg/mL each of histidine, tryptophan, and adenine, and 0.06 mg/ml of leucine. Another non-limiting example of a medium suitable for the culture of Yeast immunotherapy composition of the invention is UL2 medium. UL2 medium comprises the following components: 15 g/L of glucose, 6.7 g/L of Yeast nitrogen base containing ammonium sulfate, and 0.04 mg/mL each of histidine, tryptophan, and adenine.

The pH level is important in the culturing of yeast. One of skill in the art will appreciate that the culturing process includes not only the start of the yeast culture but the maintenance of the culture as well. The yeast culture may be started at any pH level, however, since the media of a yeast culture tends to become more acidic (i.e., lowering the pH) over time, care must be taken to monitor the pH level during the culturing process.

In some aspects of the invention, the yeast is grown in a media at a pH level of at least 5.5. In other aspects, the yeast is grown at a pH level of about 5.5. In other aspects, the yeast is grown at a pH level of between 5.5 and 8. In some cases, the yeast culture is maintained at a pH level of between 5.5 and 8. In other aspects, the yeast is grown at a pH level of between 6 and 8. In some cases, the yeast culture is maintained at a pH level of between 6 and 8. In other aspects, the yeast is grown and/or maintained at a pH level of between 6.1 and 8.1. In other aspects, the yeast is grown and/or maintained at a pH level of between 6.2 and 8.2. In other aspects, the yeast is grown and/or maintained at a pH level of between 6.3 and 8.3. In other aspects, the yeast is grown and/or maintained at a pH level of between 6.4 and 8.4. In other aspects, the yeast is grown and/or maintained at a pH level of between 5.5 and 8.5. In other aspects, the yeast is grown and/or maintained at a pH level of between 6.5 and 8.5. In other aspects, the yeast is grown at a pH level of about 5.6, 5.7, 5.8 or 5.9. In another aspect, the yeast is grown at a pH level of about 6. In another aspect, the yeast is grown at a pH level of about 6.5. In other aspects, the yeast is grown at a pH level of about 6, 6.1, 6.2, 6.3, 6.4, 6.5, 6.6, 6.7, 6.8, 6.9 or 7.0. In other aspects, the yeast is grown at a pH level of about 7, 7.1, 7.2, 7.3, 7.4, 7.5, 7.6, 7.7, 7.8, 7.9, or 8.0. In other aspects, the yeast is grown at a level of above 8. As discussed above, in some embodiments, the yeast can be cultured at neutral pH to produce yeast believed to be more effective at modulating MDSC suppressive function and differentiation.

As yeast grow and replicate, the cell densities become greater and the acidity level in the culture media rises. As such, it is recommended that as the yeast are cultured at a pH level of at least 5.5 and/or maintained at least pH 5.5 as the yeast density increases. In one aspect, the yeast are grown and/or maintained between a pH of 5.5 and 8 as the yeast density is 0.5 yeast units (YU)/ml or above. In other aspects, the yeast are grown and/or maintained between a pH of 5.5 and 8 when the yeast density is at least 0.6 YU/ml or above, preferably 0.7 YU/ml or above, 0.8 YU/ml or above, 0.9 YU/ml or above, or 1 YU/ml or above. In another aspect, the yeast are grown and/or maintained between a pH of 6 and 8 as the yeast density is 0.5 YU/ml or above. In other aspects, the yeast are grown and/or maintained between a pH of 6 and 8 when the yeast density is at least 0.6 YU/ml or above, preferably 0.7 YU/ml or above, 0.8 YU/ml or above, 0.9 YU/ml or above, or 1 YU/ml or above.

In some aspects, it is preferable at the time of harvest that the yeast culture is at a neutral pH level. In some cases, the yeast culture, at the time of harvest, will be at a pH level of between 6 and 8. In other cases, the yeast culture, at the time of harvest, will be at a pH level of between 5.5 and 8.

The culture media can be brought to a pH level of at least 5.5 by any means. In one aspect, succinic acid (and any related forms, e.g., the anion succinate) is used for buffering the culture media. As further detailed in the Examples, the use of succinate to buffer the culture media to at least pH 5.5 allows for yeast to have a doubling time of about two to two and a half hours. Succinate is available from commercially available sources (e.g., Sigma Chemicals). In other aspects, citrate may be used to bring the media to a pH of at least 5.5. One of skill in the art will be able to readily determine other buffering agents which may be used to bring the media to a pH of at least 5.5 while keeping the yeast viable. The concept of buffering agents to keep a solution at a steady pH level is well-known in the art and as such, will not be discussed in detail herein. If yeast grown according to the invention are being used for pharmaceutical formulations (e.g., vaccines), it is recommended that GMP grade material be used.

In addition, other supplements may be added to the culture media to improve the media. Other supplements which are particularly helpful to add to the culture media include soytone. Soytone is readily available from commercial sources (e.g., BD Difco). As shown in the Examples and figures, the addition of soytone to the culture media supports higher density for growth at neutral pH. Furthermore, the addition of soytone supports expression of an antigen of interest.

Other additives may be added to the yeast culture for other purposes, such as inducing expression of heterologous genes. In some aspects, copper is used to induce the expression of hemagglutinin. However, the use of copper is not ideal at neutral pH thus, for control of inducible genes to be expressed in yeast grown at neutral pH; an additive other than copper would be recommended.

In one embodiment of the present invention, as an alternative to expression of an antigen or other protein recombinantly in the yeast, a yeast is loaded intracellularly with the protein or peptide, or with carbohydrates or other molecules that serve as an antigen and/or are useful as immunomodulatory agents or biological response modifiers according to the invention. Subsequently, the yeast, which now contains the antigen and/or other proteins intracellularly, can be administered to an individual or loaded into a carrier such as a dendritic cell. Peptides and proteins can be inserted directly into yeast of the present invention by techniques known to those skilled in the art, such as by diffusion, active transport, liposome fusion, electroporation, phagocytosis, freeze-thaw cycles and bath sonication. Yeast that can be directly loaded with peptides, proteins, carbohydrates, or other molecules include intact yeast, as well as spheroplasts, ghosts or cytoplasts, which can be loaded with antigens and other agents after production. Alternatively, intact yeast can be loaded with the antigen and/or agent, and then spheroplasts, ghosts, cytoplasts, or subcellular particles can be prepared therefrom. Any number of antigens and/or other agents can be loaded into a yeast in this embodiment, from at least 1, 2, 3, 4 or any whole integer up to hundreds or thousands of antigens and/or other agents, such as would be provided by the loading of a microorganism or portions thereof, for example.

In another embodiment of the present invention, an antigen and/or other agent is physically attached to the yeast. Physical attachment of the antigen and/or other agent to the yeast can be accomplished by any method suitable in the art, including covalent and non-covalent association methods which include, but are not limited to, chemically crosslinking the antigen and/or other agent to the outer surface of the yeast or biologically linking the antigen and/or other agent to the outer surface of the yeast, such as by using an antibody or other binding partner. In one aspect, antigen(s) and/or agents (including immuno-modulating antibodies) are expressed on the yeast cell wall covalently by fusing them in frame to outer cell wall proteins. Chemical cross-linking can be achieved, for example, by methods including glutaraldehyde linkage, photoaffinity labeling, treatment with carbodiimides, treatment with chemicals capable of linking di-sulfide bonds, and treatment with other cross-linking chemicals standard in the art. Alternatively, a chemical can be contacted with the yeast that alters the charge of the lipid bilayer of yeast membrane or the composition of the cell wall so that the outer surface of the yeast is more likely to fuse or bind to antigens and/or other agent having particular charge characteristics. Targeting agents such as antibodies, binding peptides, soluble receptors, and other ligands may also be incorporated into an antigen as a fusion protein or otherwise associated with an antigen for binding of the antigen to the yeast.

When the antigen or other protein is expressed on or physically attached to the surface of the yeast, spacer arms may, in one aspect, be carefully selected to optimize antigen or other protein expression or content on the surface. The size of the spacer arm(s) can affect how much of the antigen or other protein is exposed for binding on the surface of the yeast. Thus, depending on which antigen(s) or other protein(s) are being used, one of skill in the art will select a spacer arm that effectuates appropriate spacing for the antigen or other protein on the yeast surface. In one embodiment, the spacer arm is a yeast protein of at least 450 amino acids. Spacer arms have been discussed in detail above.

In yet another embodiment, the yeast and the antigen or other protein are associated with each other by a more passive, non-specific or non-covalent binding mechanism, such as by gently mixing the yeast and the antigen or other protein together in a buffer or other suitable formulation (e.g., admixture).

In one embodiment, intact yeast (with or without expression of heterologous antigens or other proteins) can be ground up or processed in a manner to produce yeast cell wall preparations, yeast membrane particles or yeast fragments (i.e., not intact) and the yeast fragments can, in some embodiments, be provided with or administered with other compositions that include antigens (e.g., DNA vaccines, protein subunit vaccines, killed or inactivated pathogens, viral vector vaccines) to enhance immune responses. For example, enzymatic treatment, chemical treatment or physical force (e.g., mechanical shearing or sonication) can be used to break up the yeast into parts that are used as an adjuvant.

In one embodiment of the invention, yeast useful in the invention include yeast that have been killed or inactivated. Killing or inactivating of yeast can be accomplished by any of a variety of suitable methods known in the art. For example, heat inactivation of yeast is a standard way of inactivating yeast, and one of skill in the art can monitor the structural changes of the target antigen, if desired, by standard methods known in the art. Alternatively, other methods of inactivating the yeast can be used, such as chemical, electrical, radioactive or UV methods. See, for example, the methodology disclosed in standard yeast culturing textbooks such as Methods of Enzymology, Vol. 194, Cold Spring Harbor Publishing (1990). Any of the inactivation strategies used should take the secondary, tertiary or quaternary structure of the target antigen into consideration and preserve such structure as to optimize its immunogenicity.

Yeast can be formulated into yeast-based immunotherapy compositions or products of the present invention using a number of techniques known to those skilled in the art. For example, yeast can be dried by lyophilization. Formulations comprising yeast can also be prepared by packing yeast in a cake or a tablet, such as is done for yeast used in baking or brewing operations. In addition, yeast can be mixed with a pharmaceutically acceptable excipient, such as an isotonic buffer that is tolerated by a host or host cell. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, glycerol or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

Composition with Unmasked Yeast

Another embodiment of the invention is a pharmaceutical composition comprising a yeast that has been modified to unmask yeast cell wall β-glucans and a pharmaceutically acceptable excipient. Such yeast have been described above and include yeast that are genetically engineered to reduce the enzymatic activity of enzymes that elongate and/or branch yeast cell wall mannosyl groups, as well as, yeast that are genetically engineered to reduce the enzymatic activity of a Golgi mannosyltransferase complex. Such modifications can also include yeast that are genetically engineered to increase the enzymatic activity of enzymes that produce β-glucans in yeast. Such yeast also include yeast that have been exposed to PNGaseF or ENDO-h to remove N-linked mannosyl groups from cell wall glycoproteins or alpha-mannosidase which removes O-linked oligosaccharides from cell wall glycoproteins.

Pharmaceutically acceptable excipients can include an isotonic buffer that is tolerated by a host or host cell. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, glycerol or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

In a further embodiment of the pharmaceutical composition of the invention, the yeast modified to unmask yeast cell wall β-glucans in the pharmaceutical composition can have been cultured can have been cultured under conditions of neutral pH as described above.

In a further embodiment of the pharmaceutical composition of the invention, the composition comprises a cancer antigen as generally described above, such as a heterologous antigen expressed by yeast.

### Use of Additional Agents

Further embodiments of the invention include the use of agents in addition to those described above and compositions comprising such additional agents, which may also be referred to as biological response modifier compounds. For example, a yeast of the invention (whether or not transfected with or loaded with at least one antigen) can be transfected or loaded with at least one agent/biological response modifier compound, or a composition of the invention can be administered in conjunction with at least one agent/biological response modifier. Biological response modifiers include adjuvants and other compounds that can modulate immune responses, which may be referred to as immunomodulatory compounds, as well as compounds that modify the biological activity of another compound or agent, such as a yeast-based therapeutic of the invention, such biological activity not being limited to immune system effects. Certain immunomodulatory compounds can stimulate a protective immune response whereas others can suppress a harmful immune response, and whether an immunomodulator is useful in combination with a given yeast-based immunotherapeutic may depend, at least in part, on the disease state or condition to be treated or prevented, and/or on the individual who is to be treated. Certain biological response modifiers preferentially enhance a cell-mediated immune response whereas others preferentially enhance a humoral immune response (i.e., can stimulate an immune response in which there is an increased level of cell-mediated compared to humoral immunity, or vice versa.). Certain biological response modifiers have one or more properties in common with the biological properties of yeast-based therapeutics or enhance or complement the biological properties of yeast-based therapeutics. There are a number of techniques known to those skilled in the art to measure stimulation or suppression of immune responses, as well as to differentiate cell-mediated immune responses from humoral immune responses, and to differentiate one type of cell-mediated response from another (e.g., a TH17 response versus a TH1 response).

Agents/biological response modifiers useful in the invention may include, but are not limited to, cytokines, chemokines, hormones, lipidic derivatives, peptides, proteins, polysaccharides, small molecule drugs, antibodies and antigen binding fragments thereof (including, but not limited to, anti-cytokine antibodies, anti-cytokine receptor antibodies, anti-chemokine antibodies), vitamins, polynucleotides, nucleic acid binding moieties, aptamers, and growth modulators. Some suitable agents include, but are not limited to, IL-1 or agonists of IL-1 or of IL-1R, anti-IL-1 or other IL-1 antagonists; IL-6 or agonists of IL-6 or of IL-6R, anti-IL-6 or other IL-6 antagonists; IL-12 or agonists of IL-12 or of IL-12R, anti-IL-12 or other IL-12 antagonists; IL-17 or agonists of IL-17 or of IL-17R, anti-IL-17 or other IL-17 antagonists; IL-21 or agonists of IL-21 or of IL-21R, anti-IL-21 or other IL-21 antagonists; IL-22 or agonists of IL-22 or of IL-22R, anti-IL-22 or other IL-22 antagonists; IL-23 or agonists of IL-23 or of IL-23R, anti-IL-23 or other IL-23 antagonists; IL-25 or agonists of IL-25 or of IL-25R, anti-IL-25 or other IL-25 antagonists; IL-27 or agonists of IL-27 or of IL-27R, anti-IL-27 or other IL-27 antagonists; type I interferon (including IFN-α) or agonists or antagonists of type I interferon or a receptor thereof; type II interferon (including IFN-γ) or agonists or antagonists of type II interferon or a receptor thereof; anti-CD40, CD40L, lymphocyte-activation gene 3 (LAG3) protein and/or IMP321 (T-cell immunostimulatory factor derived from the soluble form of LAG3), anti-CTLA-4 antibody (e.g., to release anergic T cells); T cell co-stimulators (e.g., anti-CD137, anti-CD28, anti-CD40); alemtuzumab (e.g., CAMPATH®), denileukin diftitox (e.g., ONTAK®); anti-CD4; anti-CD25; anti-PD-1, anti-PD-L1, anti-PD-L2; agents that block FOXP3 (e.g., to abrogate the activity/kill CD4+/CD25+T regulatory cells); Flt3 ligand, imiquimod (Aldara™), granulocyte-macrophage colony stimulating factor (GM-CSF); granulocyte-colony stimulating factor (G-CSF), sargramostim (LEUKINE®); hormones including without limitation prolactin and growth hormone; Toll-like receptor (TLR) agonists, including but not limited to TLR-2 agonists, TLR-4 agonists, TLR-7 agonists, and TLR-9 agonists; TLR antagonists, including but not limited to TLR-2 antagonists, TLR-4 antagonists, TLR-7 antagonists, and TLR-9 antagonists; anti-inflammatory agents and immunomodulators, including but not limited to, COX-2 inhibitors (e.g., Celecoxib, NSAIDS), glucocorticoids, statins, and thalidomide and analogues thereof including IMiD™s (which are structural and functional analogues of thalidomide (e.g., REVLIMID® (lenalidomide), ACTIMID® (pomalidomide)); proinflammatory agents, such as fungal or bacterial components or any proinflammatory cytokine or chemokine; immunotherapeutic vaccines including, but not limited to, virus-based vaccines, bacteria-based vaccines, or antibody-based vaccines; and any other immunomodulators, immunopotentiators, anti-inflammatory agents, pro-inflammatory agents, and any agents that modulate the number of, modulate the activation state of, and/or modulate the survival of antigen-presenting cells or of TH17, TH1, and/or Treg cells. Any combination of such agents is contemplated by the invention, and any of such agents combined with or administered in a protocol with (e.g., concurrently, sequentially, or in other formats with) a yeast-based immunotherapeutic is a composition encompassed by the invention. Such agents are well known in the art. In the case of administration of immunotherapeutic compositions, such compositions may include, but are not limited to, yeast-based immunotherapy, recombinant virus-based immunotherapy (viral vectors, e.g., see PCT Publication No. WO/00/34494), cytokine therapy, immunostimulant therapy (including chemotherapy with immuno-stimulating properties), DNA vaccines, dendritic cell/tumor fusion immunotherapy (e.g., see PCT Publication No. WO/2009/062001), and other immunotherapy compositions. These agents may be used alone or in combination with other agents described herein.

A virus-based immunotherapy composition typically comprises a viral vector comprising a virus genome or portions thereof (e.g., a recombinant virus) and a nucleic acid sequence encoding at least one antigen(s) from a disease-causing agent or disease state (e.g., a cancer antigen(s), and/or at least one immunogenic domain thereof). In some embodiments, a virus-based immunotherapy composition further includes at least one viral vector comprising one or more nucleic acid sequences encoding one or more immunostimulatory molecule(s). In some embodiments, the genes encoding immunostimulatory molecules and antigens are inserted into the same viral vector (the same recombinant virus).

Dendritic cell/tumor cell fusion immunotherapy compositions typically are hybrid cells generated by the fusion between dendritic cells and non-dendritic cells that express tumor antigens, including tumor cells, using fusion methods that are known in the art. The fused cells have dendritic cell characteristics and also express and present tumor antigens from the tumor cell. The compositions may be administered to an individual or used to stimulate T cells ex vivo for T cell transfer methods.

Agents can include agonists and antagonists of a given protein or peptide or domain thereof. As used herein, an “agonist” is any compound or agent, including without limitation small molecules, proteins, peptides, antibodies, nucleic acid binding agents, etc., that binds to a receptor or ligand and produces or triggers a response, which may include agents that mimic or enhance the action of a naturally occurring substance that binds to the receptor or ligand. An “antagonist” is any compound or agent, including without limitation small molecules, proteins, peptides, antibodies, nucleic acid binding agents, etc., that blocks or inhibits or reduces the action of an agonist.

Compositions of the invention can further include or can be administered with (concurrently, sequentially, or intermittently with) any other agents or compositions or protocols that are useful for preventing or treating cancer or any compounds that treat or ameliorate any symptom of cancer. In addition, compositions of the invention can be used together with other immunotherapeutic compositions, including prophylactic and/or therapeutic immunotherapy. Additional agents, compositions or protocols (e.g., therapeutic protocols) that are useful for the treatment of cancer include, but are not limited to, chemotherapy, surgical resection of a tumor, radiation therapy, allogeneic or autologous stem cell transplantation, T cell adoptive transfer, other types of immunotherapy, including viral vector-based immunotherapy and dendritic cell/tumor fusion immunotherapy, and/or targeted cancer therapies (e.g., small molecule drugs, biologics, or monoclonal antibody therapies that specifically target molecules involved in tumor growth and progression, including, but not limited to, selective estrogen receptor modulators (SERMs), aromatase inhibitors, tyrosine kinase inhibitors, serine/threonine kinase inhibitors, histone deacetylase (HDAC) inhibitors, retinoid receptor activators, apoptosis stimulators, angiogenesis inhibitors, poly (ADP-ribose) polymerase (PARP) inhibitors, or immuno-stimulators). Any of these additional therapeutic agents and/or therapeutic protocols may be administered before, concurrently with, alternating with, or after the compositions of the invention, or at different time points. For example, when given to an individual in conjunction with chemotherapy or a targeted cancer therapy, it may be desirable to administer the yeast compositions during the “holiday” between doses of chemotherapy or targeted cancer therapy, in order to maximize the efficacy of the immunotherapy compositions. Surgical resection of a tumor may frequently precede administration of a yeast therapy composition, but additional or primary surgery may occur during or after administration of a yeast therapy composition.

The invention also includes a kit comprising any of the compositions described herein, or any of the individual components of the compositions described herein. Kits may include additional reagents and written instructions or directions for using any of the compositions of the invention to prevent or treat cancer.

### General Techniques Useful in the Invention

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Methods of Enzymology, Vol. 194, Guthrie et al., eds., Cold Spring Harbor Laboratory Press (1990); Biology and activities of yeasts, Skinner, et al., eds., Academic Press (1980); Methods in yeast genetics: a laboratory course manual, Rose et al., Cold Spring Harbor Laboratory Press (1990); The Yeast Saccharomyces: Cell Cycle and Cell Biology, Pringle et al., eds., Cold Spring Harbor Laboratory Press (1997); The Yeast Saccharomyces: Gene Expression, Jones et al., eds., Cold Spring Harbor Laboratory Press (1993); The Yeast Saccharomyces: Genome Dynamics, Protein Synthesis, and Energetics, Broach et al., eds., Cold Spring Harbor Laboratory Press (1992); Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988), Antibodies, A Laboratory Manual, Cold Spring Harbor Publications, New York; Harlow and Lane (1999) Using Antibodies: A Laboratory Manual, Cold Spring Harbor Laboratory Press, Cold Spring Harbor, N.Y. (jointly referred to herein as “Harlow and Lane”), Beaucage et al. eds., Current Protocols in Nucleic Acid Chemistry, John Wiley & Sons, Inc., New York, 2000); Casarett and Doull's Toxicology The Basic Science of Poisons, C. Klaassen, ed., 6th edition (2001), and Vaccines, S. Plotkin, W. Orenstein, and P. Offit, eds., Fifth Edition (2008).

### General Definitions

A “TARMOGEN®” (GlobeImmune, Inc., Louisville, Colo.) generally refers to a yeast expressing one or more heterologous antigens extracellularly (on its surface), intracellularly (internally or cytosolically) or both extracellularly and intracellularly. TARMOGEN®s have been generally described (see, e.g., U.S. Pat. No. 5,830,463). Certain yeast-based immunotherapy compositions, and methods of making and generally using the same, are also described in detail, for example, in U.S. Pat. Nos. 5,830,463, 7,083,787, 7,736,642, Stubbs et al., Nat. Med. 7:625-629 (2001), Lu et al., Cancer Research 64:5084-5088 (2004), and in Bernstein et al., Vaccine 2008 January 24;26(4):509-21, each of which is incorporated herein by reference in its entirety.

As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance but has a different structure or origin with respect to the reference compound.

The terms “substituted”, “substituted derivative” and “derivative”, when used to describe a compound, means that at least one hydrogen bound to the unsubstituted compound is replaced with a different atom or a chemical moiety.

Although a derivative has a similar physical structure to the parent compound, the derivative may have different chemical and/or biological properties than the parent compound. Such properties can include, but are not limited to, increased or decreased activity of the parent compound, new activity as compared to the parent compound, enhanced or decreased bioavailability, enhanced or decreased efficacy, enhanced or decreased stability in vitro and/or in vivo, and/or enhanced or decreased absorption properties.

In general, the term “biologically active” indicates that a compound (including a protein or peptide) has at least one detectable activity that has an effect on the metabolic, physiological, chemical, or other processes of a cell, a tissue, or an organism, as measured or observed in vivo (i.e., in a natural physiological environment) or in vitro (i.e., under laboratory conditions).

According to the present invention, the term “modulate” can be used interchangeably with “regulate” and refers generally to upregulation or downregulation of a particular activity. As used herein, the term “upregulate” can be used generally to describe any of: elicitation, initiation, increasing, augmenting, boosting, improving, enhancing, amplifying, promoting, or providing, with respect to a particular activity. Similarly, the term “downregulate” can be used generally to describe any of: decreasing, reducing, inhibiting, ameliorating, diminishing, lessening, blocking, or preventing, with respect to a particular activity.

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

According to the present invention, the phrase “selectively binds to” refers to the ability of an antibody, antigen-binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art including enzyme immunoassays (e.g., ELISA, immunoblot assays, etc.).

General reference to a protein or polypeptide used in the present invention includes full-length proteins, or any fragment, domain (structural, functional, or immunogenic), conformational epitope, or a homologue or variant of a given protein. A fusion protein may also be generally referred to as a protein or polypeptide. An isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. According to the present invention, the terms “modification” and “mutation” can be used interchangeably, particularly with regard to the modifications/mutations to the amino acid sequence of proteins or portions thereof (or nucleic acid sequences) described herein.

As used herein, the term “homologue” or “variant” is used to refer to a protein or peptide which differs from a reference protein or peptide (i.e., the “prototype” or “wild-type” protein) by minor modifications to the reference protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue or variant can have enhanced, decreased, or substantially similar properties as compared to the reference protein or peptide. A homologue or variant can include an agonist of a protein or an antagonist of a protein. Homologues or variants can be produced using techniques known in the art for the production of proteins including, but not limited to, direct modifications to the isolated reference protein, direct protein synthesis, or modifications to the nucleic acid sequence encoding the protein using, for example, classic or recombinant DNA techniques to effect random or targeted mutagenesis, resulting in the encoding of a protein variant. In addition, naturally occurring variants of a reference protein may exist (e.g., isoforms, allelic variants, or other natural variants that may occur from individual to individual) and may be isolated, produced and/or utilized in the invention.

A homologue or variant of a given protein may comprise, consist essentially of, or consist of, an amino acid sequence that is at least about 45%, or at least about 50%, or at least about 55%, or at least about 60%, or at least about 65%, or at least about 70%, or at least about 75%, or at least about 80%, or at least about 85%, or at least about 86% identical, or at least about 87% identical, or at least about 88% identical, or at least about 89% identical, or at least about 90%, or at least about 91% identical, or at least about 92% identical, or at least about 93% identical, or at least about 94% identical, or at least about 95% identical, or at least about 96% identical, or at least about 97% identical, or at least about 98% identical, or at least about 99% identical (or any percent identity between 45% and 99%, in whole integer increments), to the amino acid sequence of the reference protein (e.g., an amino acid sequence specified herein, or the amino acid sequence of a specified protein). In one embodiment, the homologue or variant comprises, consists essentially of, or consists of, an amino acid sequence that is less than 100% identical, less than about 99% identical, less than about 98% identical, less than about 97% identical, less than about 96% identical, less than about 95% identical, and so on, in increments of 1%, to less than about 70% identical to the amino acid sequence of the reference protein.

As used herein, unless otherwise specified, reference to a percent (%) identity refers to an evaluation of homology which is performed using: (1) a Basic Local Alignment Search Tool (BLAST) basic homology search using blastp for amino acid searches and blastn for nucleic acid searches with standard default parameters, wherein the query sequence is filtered for low complexity regions by default (such as described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST alignment of two sequences (e.g., using the parameters described below); β) and/or PSI-BLAST with the standard default parameters (Position-Specific Iterated BLAST. It is noted that due to some differences in the standard parameters between Basic BLAST and BLAST for two sequences, two specific sequences might be recognized as having significant homology using the BLAST program, whereas a search performed in Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST as described in Tatusova and Madden, (1999), “Blast 2 sequences—a new tool for comparing protein and nucleotide sequences”, FEMS Microbiol Lett. 174:247-250, incorporated herein by reference in its entirety. Such a sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST sequence alignment for two sequences is performed using the standard default parameters as follows.
For blastn, using 0 BLOSUM62 matrix:
Reward for match=1
Penalty for mismatch=−2
Open gap (5) and extension gap (2) penalties
gap x_dropoff (50) expect (10) word size (11) filter (on)
For blastp, using 0 BLOSUM62 matrix:
Open gap (11) and extension gap (1) penalties
gap x_dropoff (50) expect (10) word size (3) filter (on).

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified but indicates that the molecule does not include an entire genome or an entire chromosome or a segment of the genome containing more than one gene, in which the nucleic acid molecule is found in nature. An isolated nucleic acid molecule can include a complete gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule may also include portions of a gene. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a plasmid useful for transfecting yeast. Recombinant vectors can be used in the cloning, sequencing, and/or otherwise manipulating of nucleic acid molecules, and can be used in delivery of such molecules (e.g., as in a DNA composition or a viral vector-based composition). Recombinant vectors are preferably used in the expression of nucleic acid molecules and can also be referred to as expression vectors. Preferred recombinant vectors are capable of being expressed in a transfected host cell, such as a yeast.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast. In microbial systems, the term “transformation” is used to describe an inherited change due to the acquisition of exogenous nucleic acids by the microorganism and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

The following experimental results are provided for purposes of illustration and are not intended to limit the scope of the invention.

### EXAMPLES

The experiments below use the basic mammary cell adenocarcinoma model (E0771) as described in FIG. 1 of Albeituni et al. (2016). Tumors are implanted and at day 8 post implantation the mice are fed with commercial β-glucan particles or various yeast preparations as described in the tables below. Although several flow cytometry readouts can be used as surrogates of the effect of β-glucans on MDSCs, the effects on tumor growth and spleen enlargement are first determined as these are the functional readouts.

In animals where differences in tumor size exist between PBS-fed vs. β-glucan (or yeast) fed animals, flow analysis is done to evaluate of the frequency of M-MDSC and PMN-MDSC cells in the treatment groups.

### Example 1

This example describes the effect of commercial 1,3 β-glucan or yeast (1 YU vs. 10 YU) on MDSCs and tumor growth). Yeast cells tested include a standard yeast preparation that is grown in unbuffered conventional yeast growth medium, or a preparation of yeast cultured in a buffered, neutral pH medium as described above (hereafter referred to as “DEC yeast”).

TABLE 1

| Treatment group | Tumor Implantation | Oral gavage |
|---|---|---|
| (n) | (Day 0) (SC) | (Day 8) |
| A (10) | 600,000 E0771 | PBS |
| B (10) | 600,000 E0771 | 800 ug particulate β-glucan |
| | | (Biothera) |
| C (10) | 600,000 E0771 | W303α standard yeast 1 YU |
| D (10) | 600,000 E0771 | W303α standard yeast 10 |
| | | YU |
| E (10) | 600,000 E0771 | W303α DEC yeast 1 YU |
| F (10) | 600,000 E0771 | W303α DEC yeast 10 YU |

Tumor growth is monitored every 3 days with calipers. When the first tumors reach 10 mm in diameter, and if there is a tumor size difference between any of the yeast-treated groups versus PBS, the tumors and spleens are excised and weighed. The excised spleens and tumor cells are processed per standard methods and stained with antibodies recognizing Ly6C, ly6G, CD11b, CD45. Flow cytometric analysis is run and determination of gating strategies for defining M-MDSC and PMN-MDSC is done followed by the creation of plots and statistical analysis.

The results will show that the yeast-based treatments achieve smaller tumor size and/or reduced levels of Ly6C, ly6G, CD11b, and/or CD45 and/or reduced numbers of M-MDSCs and/or PMNCs as compared to the PBS control.

### Example 2

This example describes the effect of commercial 1,3 b-glucan and compares against wild-type (WT) yeast, mnn10 mutant yeast and PNGase F—treated yeast (either a standard or DEC preparation). The optimal dose of the yeast is determined from the results in Example 1.

TABLE 2

| Treatment group | Tumor Implantation | Oral gavage |
|---|---|---|
| (n) | (Day 0) (SC) | (Day 8) |
| A (10) | 600,000 E0771 | PBS |
| B (10) | 600,000 E0771 | 800 μg particulate b-glucan |
| | | (Biothera) |
| C (10) | 600,000 E0771 | W303α WT yeast (std or DEC) |
| D (10) | 600,000 E0771 | mnn10 mutant yeast (std or |
| | | DEC) |
| E (10) | 600,000 E0771 | PNGase-F treated yeast (std or |
| | | DEC) |

Tumor growth is monitored every 3 days with calipers. When the first tumors reach 10 mm in diameter, and if there is a difference between any yeast and PBS, the tumors and spleens are excised and weighed. The excised spleens and tumor cells are processed per standard methods and stained with antibodies recognizing Ly6C, ly6G, CD11b, CD45. Flow cytometric analysis is run and determination of gating strategies for defining M-MDSC and PMN-MDSC is done followed by the creation of plots and statistical analysis.

The results will show that treatments of the yeast cells that unmask the cell wall β-glucan layer (mnn10 deletion or PNGaseF treatment) result in smaller tumor size and/or reduced levels of Ly6C, ly6G, CD11b, and/or CD45 and/or reduced numbers of M-MDSCs and/or PMNCs as compared to mice injected with PBS (Group A) or untreated WT yeast (group C).

### Example 3

This example is similar to Examples 1 and 2 above except that instead of oral gavage as the administration route, the administration route is intratumoral injection.

TABLE 3

| Treatment group | Tumor Implantation | Intratumoral injection |
|---|---|---|
| (n) | (Day 0) (SC) | (Day 8) |
| A (10) | 600,000 E0771 | PBS |
| B (10) | 600,000 E0771 | particulate β-glucan at 2 |
| | | sites on tumor (Biothera) |
| C (10) | 600,000 E0771 | W303α standard yeast 10 |
| | | YU (5 YU at 2 sites on |
| | | tumor) |
| D (10) | 600,000 E0771 | W303α DEC yeast 10 YU (5 |
| | | YU at 2 sites on tumor) |
| E (10) | 600,000 E0771 | Known powerful TLR |
| | | agonist as positive control |

Tumor growth is monitored every 3 days with calipers. When the first tumors reach 10 mm in diameter, and if there is a difference between any yeast and PBS, the tumors and spleens are excised and weighed. The excised spleens and tumor cells are processed per standard methods and stained with antibodies recognizing Ly6C, ly6G, CD11b, CD45. Flow analysis is run and determination of gating strategies for defining M-MDSC and PMN-MDSC is done followed by the creation of plots and statistical analysis.

The results will show that the yeast-based treatments achieve smaller tumor size and/or reduced levels of Ly6C, ly6G, CD11b, and/or CD45 and/or reduced numbers of M-MDSCs and/or PMNCs as compared to the PBS control.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

1. A method to modulate myeloid-derived suppressor cell (MDSC) suppressive function, MDSC differentiation, or combinations thereof in a cancer patient, comprising: administering a yeast to a cancer patient, wherein the yeast is treated or genetically modified to increase expression of beta-glucans (β-glucans) relative to wild type yeast.

2. (canceled)

3. The method of claim 1, wherein the yeast is genetically engineered to reduce the enzymatic activity of enzymes that elongate and/or branch yeast cell wall mannosyl groups.

4. The method of claim 3, wherein the yeast is genetically engineered to reduce the enzymatic activity of a Golgi mannosyltransferase complex.

5. The method of claim 1, wherein the yeast is selected from the group consisting of a Mnn9 deleted strain, a Mnn10 deleted strain and a doubly deleted Mnn9/Mnn10 strain of yeast.

6. The method of claim 1, wherein the yeast is genetically engineered to increase the enzymatic activity of 1,3 β-glucan synthase.

7. The method of claim 1, wherein the yeast has been treated to remove yeast cell wall glycosyl groups.

8. The method of claim 7, wherein the treatment comprises contacting the yeast with Peptide-N-Glycosidase F (PNGaseF), Endoglycosidase H (ENDO-H) or alpha-mannosidase.

9. The method of claim 1, wherein the yeast was cultured in medium wherein the media was maintained at a pH level of between 5.5 and 8 for at least 50% of time that the yeast were being cultured.

10. The method of claim 1, wherein the yeast is administered to the cancer patient by oral administration.

11. The method of claim 1, wherein the yeast is administered to the cancer patient by intratumoral administration.

12. The method of claim 1, wherein the cancer patient has a cancer selected from the group consisting of lung cancer, breast cancer, triple negative breast cancer (TNBC), colorectal cancer, liver cancer, stomach cancer, colon cancer, non-small cell lung cancer (NSCLC), bone cancer, malignant chordoma, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladder cancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary central nervous system (CNS) tumor, spinal cord tumor, brainstem glioma, urothelial, merkel, head and neck squamous cell carcinoma, and pituitary adenoma.

13. (canceled)

14. The method of claim 1, wherein the yeast is a yeast vehicle selected from the group consisting of: a whole yeast, a yeast spheroplast, a yeast cytoplast, a yeast ghost, and a subcellular yeast particle; and the yeast is in an immunogenic composition further comprising at least one antigen and/or neoantigen that is heterologous to the yeast.

15. The method of claim 14, wherein the antigen is an antigen from a tumor or cancer selected from the group consisting of melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, leukemias, lymphomas, primary hepatic cancers, lung cancers, pancreatic cancers, gastrointestinal cancers, renal cell carcinomas, hematopoietic neoplasias and metastatic cancers thereof.

16. The method of claim 14, wherein the antigen is selected from the group consisting of carcinoembryonic antigen (CEA), carcinoembryonic antigen peptide 1 (CAP-1), carcinoembryonic antigen peptide 1-6D (CAP-1-6D), melanoma antigen recognized by T cells 1 (MART-1), melanoma-associated antigen 1 (MAGE-1), melanoma-associated antigen 3 (MAGE-3), GAGE, glycoportien 100 (GP-100), mucin 1 (MUC-1), mucin 2 (MUC-2), point mutated Ras oncoprotein, tumor protein p53 (p53), point mutated p53, prostate-specific membrane antigen (PSMA), tyrosinase, tyrosinase related protein 1 (TRP-1), New York esophageal squamous cells carcinoma 1 (NY-ESO-1), tyrosinase related protein 2 (TRP-2), tumor associated glycoprotein 72 (TAG72), KSA, cancer antigen 125 (CA-125), prostate-specific antigen (PSA), human epidermal growth factor receptor 2 c-erb-B2 (HER-2/neu/c-erb/B2), epidermal growth factor receptor (EGFR), human telomerase reverse transcriptase (hTERT), tumor protein 73 (p73), serine/threonine-protein kinase B-Raf (B-RAF), adenomatous polyposis coli (APC), Myc, von Hippel-Lindau protein (VHL), retinoblastoma protein 1 (Rb-1), retinoblastoma protein 2 (Rb-2), androgen receptor (AR), mothers against decapentaplegic homolog 4 (Smad4), multi-drug resistance 1 (MDR1), FMS-like tyrosine kinase 3 (Flt-3), breast cancer gene 1 (BRCA-1), breast cancer gene 2 (BRCA-2), Bcr-Abl, pax3-fkhr, ews-fli-1, Brachyury, human endogenous retrovirus subfamily H (HERV-H), human endogenous retrovirus subfamily K (HERV-K), TWIST, Mesothelin, new gene expressed in prostate (NGEP), and modifications and epitopes of such antigens.

17. The method of claim 1, wherein the yeast are from Saccharomyces.

18. The method of claim 17, wherein the yeast are from Saccharomyces cerevisiae.

19. The method of claim 1, wherein administration of yeast has one or more effects selected from the group consisting of reducing, limiting or inhibiting MDSC suppression of NK cells, MDSC induction of regulatory T cells, MDSC maturation to tumor-associated macrophages, and combinations thereof.

20. The method of claim 1, wherein the modulation of MDSC suppressive function and differentiation comprises one or more effects selected from the group consisting of inducing polymorphonuclear (PMN) MDSC apoptosis, monocytic (M) MDSC differentiation to antigen presenting cells (APCs), and combinations thereof.

21. The method of claim 1, wherein administration of yeast induces apoptosis of PMN-MDSCs.

22. The method of claim 1, wherein administration of yeast induces differentiation of M-MDSC to functionally active APCs.

23. A pharmaceutical composition, comprising a yeast treated or genetically modified to increase expression of yeast cell wall β-glucans relative to wild type yeast, and a pharmaceutically acceptable excipient.

24-29. (canceled)
